(19) United States
(12) Patent Application Publication
(10) Pub. No.: US 2017/0089865 A1
(43) Pub. Date: Mar. 30, 2017

(54) Integrated Molecular Sensor System

(71) Applicant: Intel Corporation, Santa Clara, CA (US)

(72) Inventors: SASHA N. OSTER, Chandler, AZ (US); FERAS EID, Chandler, AZ (US); INDIRA NEGI, Sunnyvale, CA (US)

(21) Appl. No.: 14/865,455

(22) Filed: Sep. 25, 2015

(51) Int. Cl. G01N 29/22; G01N 33/543

(52) U.S. Cl. CPC G01N 29/22; G01N 33/54373; G01N 2291/0255; G01N 2600/00

(57) ABSTRACT

An embodiment includes a package comprising: a cavity formed in a dielectric material; a beam in the cavity; an interconnect to couple the beam to a current source; a magnet coupled to the cavity; and a polymer, on the beam, having an affinity to an analyte; wherein (a) a vertical axis intersects the magnet, the cavity, and the beam; (b) in a first state the beam and the polymer, which is not coupled to the analyte, collectively have a first mass and resonate at a first resonant frequency when the beam conducts a first current; and (c) in a second state the beam and the polymer, which is coupled to the analyte, collectively have a second mass that is greater than the first mass and resonate at a second resonant frequency when the beam conducts a second current. Other embodiments are described herein.

### TECHNICAL FIELD

Embodiments of the invention are in the field of sensors.

### BACKGROUND

The ability to detect chemicals inside and around people helps inform choices such as where a person should sit, what that person should eat, as well as longer term decisions such as where that person should live. As the world becomes more industrialized, many man-made chemical compounds and/or natural compounds are collecting in new places at ever higher concentrations. These high concentrations, or even low concentrations, may be harmful to people. To reduce the risk of this harm, chemical sensors are used to effectively monitor and/or detect the presence of chemicals both in the environment and in people/animals themselves (e.g., biomarkers in skin, expired air, saliva, blood).

### BRIEF DESCRIPTION OF THE DRAWINGS

Features and advantages of embodiments of the present invention will become apparent from the appended claims, the following detailed description of one or more example embodiments, and the corresponding figures. Where considered appropriate, reference labels have been repeated among the figures to indicate corresponding or analogous elements.

FIG. 1 shows an abstract microelectronic assembly including a chemical sensor in an embodiment of the invention.

FIGS. 2A and 2B are top and side views of a sensor, respectively, in an embodiment of the invention. FIGS. 2C-2F are top views of alternative trace geometries in embodiments of the invention.

FIG. 3 is a flowchart for making a microelectronic assembly in an embodiment of the invention.

FIG. 4 is a flowchart for using a microelectronic assembly in an embodiment of the invention.

FIGS. 5A-5G depict a method of forming a sensor in an embodiment of the invention.

FIG. 6 includes a system for use with a sensor in an embodiment of the invention.

FIGS. 7A and 7B are top and side views of a sensor, respectively, in an embodiment of the invention.

### DETAILED DESCRIPTION

Reference will now be made to the drawings wherein like structures may be provided with like suffix reference designations. In order to show the structures of various embodiments more clearly, the drawings included herein are diagrammatic representations of semiconductor/circuit structures. Thus, the actual appearance of the fabricated integrated circuit structures, for example in a photomicrograph, may appear different while still incorporating the claimed structures of the illustrated embodiments. Moreover, the drawings may only show the structures useful to understand the illustrated embodiments. Additional structures known in the art may not have been included to maintain the clarity of the drawings. For example, not every layer of a semiconductor device is necessarily shown. “An embodiment”, “various embodiments” and the like indicate embodiment(s) so described may include particular features, structures, or characteristics, but not every embodiment necessarily includes the particular features, structures, or characteristics. Some embodiments may have some, all, or none of the features described for other embodiments. “First”, “second”, “third” and the like describe a common object and indicate different instances of like objects that are being referred to. Such adjectives do not imply objects so described must be in a given sequence, either temporally, spatially, in ranking, or in any other manner. “Connected” may indicate elements are in direct physical or electrical contact with each other and “coupled” may indicate elements co-operate or interact with each other, but they may or may not be in direct physical or electrical contact.

Sensing is becoming increasingly important while computing technology is becoming increasingly ubiquitous. There is a growing demand for low cost, pervasive sensing. However, current chemical sensors often suffer from one or more shortcomings. For example, they are often bulky, expensive, lack sensitivity of the element to be sensed, consume too much power, and/or include external components that must be attached to a system on a chip (SoC) package or system board (and routed to the SOC) that performs analysis of the sensed chemical. Such sensors include, for example, photoionization detectors (PID), metal oxide semiconductor (MOS) sensors, or quartz crystals.

However, embodiments described herein address a chemical sensor with a vibrating beam, coated in a molecular imprinted polymer (MIP), whose resonant frequency shifts when the beam senses a target analyte. The beam is part of a package substrate built using a straight forward and relatively inexpensive package build-up process with unique steps (e.g., removing dielectric around a resonating beam) that help produce sensitive chemical analyte sensors. This package architecture and process enables very low cost, small sensors that embed easily into packages along with other products (e.g., a processor in a SoC that can analyze outputs from the sensor).

When compared to PID sensors, the advantages of using package-integrated MIP based sensors include lower concentration detection limits and very high selectivity, which is known to be a concern with PID sensors that typically suffer from poor selectivity. When compared to MOS sensors, the advantages of using package-integrated MIP based sensors are better sensitivity and the capability of obtaining a linear response in the concentrations of interest. When compared to quartz sensors, the advantages of using package-integrated MIP based sensors are the much smaller form factors and direct integration with the package, with no need for assembly of a discrete component. In addition, package-integrated MIP based sensors consume very low power (e.g., power consumption in the single mW range).

Thus, embodiments include a compact, mobile (e.g., wearable), affordable, real-time, reusable sensing platform with high performance and reusability for real-time sensing of low concentrations of chemical analytes (e.g., biological environmental compounds). Embodiments sense analytes with high sensitivity and selectivity, even when the analytes are present in low concentrations (e.g., on a parts per billion (ppb) level for gas analytes and a nanomole (nM) level for liquid analytes). The real-time capacity of such embodiments stands in contrast with conventional chemical sensors that are either expensive and immobile or exhibit subpar sensitivity and/or specificity.

Further, embodiments may be stand-alone products included in wearables (e.g., watches, glasses, clothing that provides data about the wearer's body (e.g., calories burned, glucose levels) and/or environment (e.g., presence of VOCs, purity of drinking water)). However, embodiments may also cooperate with computer nodes located on different substrates from the sensors such as smartphones (located on a different die or dies than the sensor). The sensors may communicate wirelessly with such a node to periodically upload data to a memory including a database or coupled to a database. The database may help a medical provider or epidemiologist track glucose levels over a period of time or exposure to specific allergens or dangerous ozone levels within the user's microclimate over a multi-day period.

Embodiments may be used for detecting dehydration (i.e., checking salt concentrations in urine or plasma), cardiopulmonary stress testing, indirect calorimetry, maximal oxygen consumption, sweat analysis, breath analysis (for exercise purposes or to gauge inebriation), and the like. An embodiment may be coupled with physical sensors (e.g., accelerometers) on the same substrate or a different substrate or within the same package. Measuring both physical and chemical information may provide for better assessment of the body's state.

An embodiment provides high sensitivity, which is required for chemical analytes originating from the body (VOCs from skin or breath). High sensitivity allows short sampling times with limited analyte volumes, which is helpful with skin gas and sweat-based monitoring.

An embodiment is usable as a fitness monitor that tracks volatile gases detectable from a human body (e.g., ketones, aldehydes, alkanes, ammonia). Such skin volatile analytes are used as biomarkers for fitness tracking. For example, acetone is used as an indicator for fat burning (one of the calorie sources) and ammonia is an indicator of dehydration.

Embodiments are now addressed in greater detail.

In one particular embodiment, a resonant beam is functionalized with a MIP. The beam is built as a part of the package substrate, with a MIP that is highly selective and sensitive to the chemical of interest to provide a low cost package embedded chemical sensor. The beam consists of a copper trace anchored by vias on one (e.g., cantilever beam) or both sides of the beam. The packaging material is then etched around the copper trace to create a free standing beam. A magnet is attached either below or above the beam or any orientation or location such that its magnetic field projects onto the beam. When an alternating current is applied across the beam at a frequency that is near the beam's resonant frequency, the induced electromagnetic force causes the beam to resonate at its natural frequency. The resonant frequency of the beam is measured and is dependent (among other factors) on the mass of the beam. In this way, in the absence of the chemical of interest, the beam has mass m0 and resonates with frequency f0. In the presence of the chemical, the chemical molecules attach to the resonant beam, increasing its mass to ml, and thereby changing its resonant frequency to f1. By measuring the change (f1-f2) in resonant frequency, the amount of chemical present is determined.

As mentioned immediately above, embodiments include beams covered with a MIP. An MIP is a chemical-selective material that has active sites for the adsorption of specific molecule types. A MIP is formed in the presence of a molecule that is extracted afterwards, thus leaving complementary cavities behind. These polymers show chemical affinity for the original molecule. When the template molecule is present in the environment the molecule attaches itself to the complementary cavity. Other molecules with different structures cannot attach to these cavities. This makes these sensors highly selective. The assembly is generally achieved by non-covalent/reversible covalent interactions, which makes these sensors reversible and reusable in various embodiments.

The method for coating the polymers differs based on the solvent for the polymer. As an example, an MIP that is created using xylene as the original template molecule is applied by dissolving the MIP in xylene and wet coating the solution on the resonant beam. The beam is then heated in air devoid of xylene, thereby activating the sensor. Once all xylene has evaporated from the MIP, the new resonant frequency of the coated beam is measured as the baseline.

Using benzene as an example, conventional PID sensors only detect concentrations in the 0-10 ppm range, have a resolution of 100 ppb, and have power requirements of ˜1-2 W due to ultraviolet (UV) lamp power consumption. Other technologies with greater sensitivity often combine multiple sensing methods but have high power requirements and longer response times (e.g., 10-15 minutes). In contrast, embodiments have a fine resolution (as small as few ppb), small size (less than 0.5 mm²), and power consumption in the mW range.

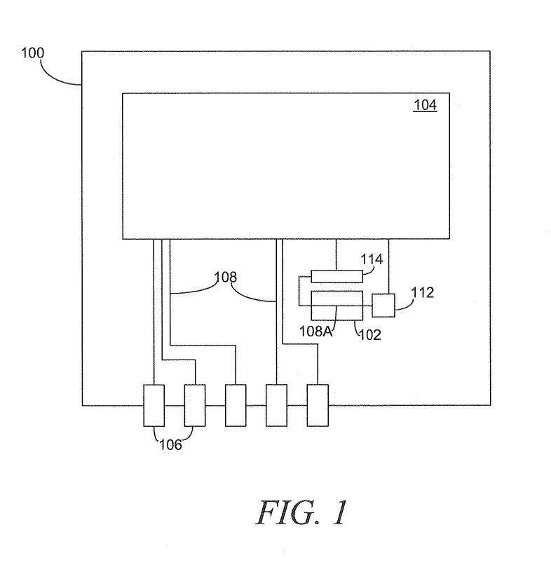

FIGS. 1 through 7A and 7B are now addressed and, in some cases, discussed simultaneously.

FIG. 1 shows an abstract microelectronic assembly 100 including a chemical sensor 102, in an example embodiment. In the illustrated example, the microelectronic assembly is a chip package, but the sensor 102 may be applied in any of a variety of microelectronic assemblies. It is emphasized that the illustration of FIG. 1 is abstract, and components are not to scale and components on different layers of the microelectronic assembly 100 are illustrated together. It is to be recognized and understood that certain structural examples are illustrated herein with particularity.

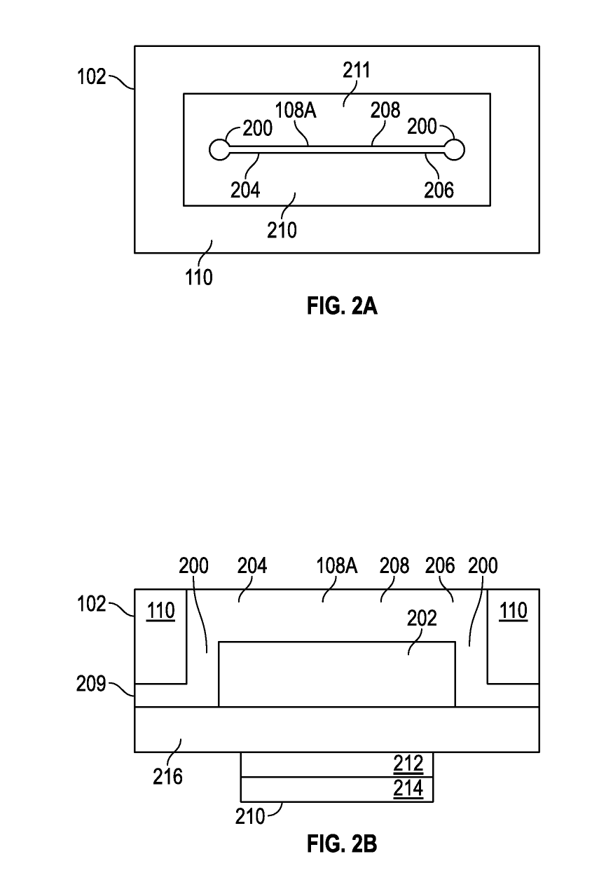

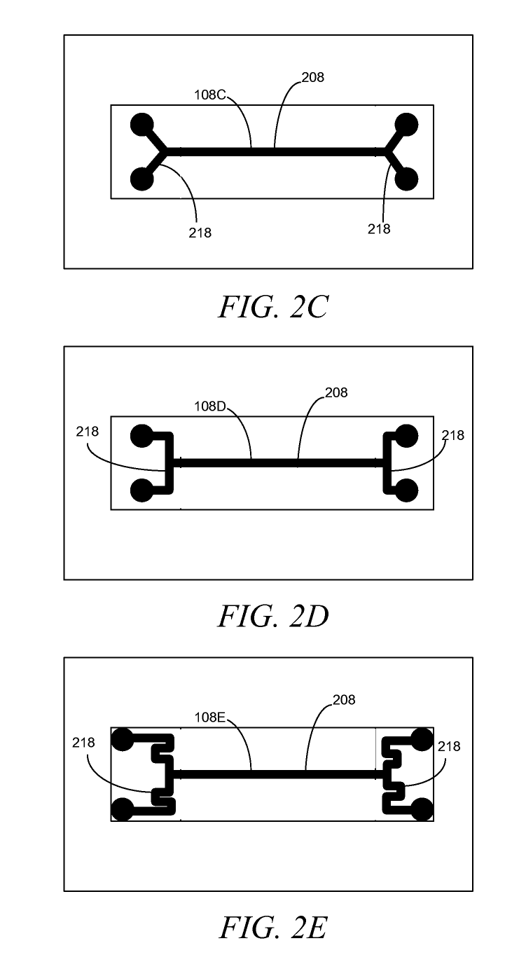

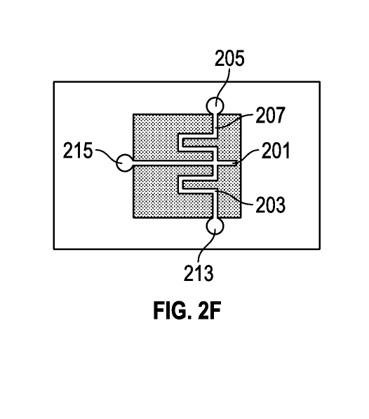

The microelectronic assembly 100 includes one or more electronic components 104, such as a silicon die, input/output terminals 106, such as pads or pins, and traces 108 to conduct electrical signals throughout the microelectronic assembly 100. The traces 108 may be formed from copper or any other suitable electrically conductive materials. The various components 104, 106, 108 of the microelectronic assembly 100 may be formed in multiple layers that are obscured from this top-down view. The components 104, 106, 108 may be electrically and mechanically isolated with respect to one another with a dielectric material 110 (FIG. 2A).

The sensor 102 includes one of the traces 108A. The MIP covered trace 108A is positioned with respect to other components of the sensor 102, as will be detailed herein. The trace 108A may variously be coupled to a current source 112 that may be or include a frequency generator configured to produce a sinusoidal current of various, selectable frequencies. The current source may be included as a component of the microelectronic assembly 100 or may be positioned outside of the microelectronic assembly 100 and is accessible through a terminal 106. A frequency detection circuit 114, such as a phase locked loop, may be positioned as a component of or proximal and connected to the sensor 102.

FIGS. 2A and 2B are top and side views of the sensor 102, respectively, in an example embodiment. The sensor 102 includes the MIP covered trace 108A secured between mechanical anchors 200. The trace 108A is partially positioned within a cavity 202 formed in the dielectric material 110. The mechanical anchors thus substantially secure a first end 204 and a second end 206 of the trace 108A while leaving a center 208 of the trace 108A free to move laterally within the cavity 202 as the trace 108A resonates, as disclosed herein.

In the illustrated example, the mechanical anchors 200 are conductive vias. The anchors 200 may secure the first end 204 and the second end 206 with respect to a package layer 209 of the electronic assembly 100, as illustrated positioned below the trace 108A. The package layer 209 may be or may include a second conductive trace and, as a result, may be electrically coupled to the trace 108A with the mechanical anchors 200 when the mechanical anchors 200 are vias or otherwise include a conductive material.

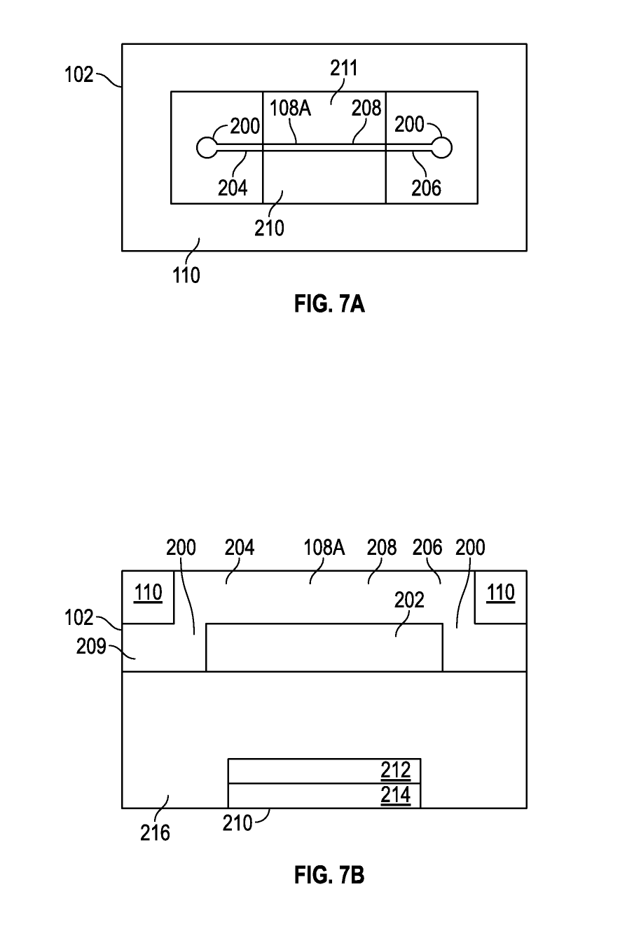

A magnet 210 is positioned with respect to the cavity 202 to induce a magnetic field 211 within the cavity 202. In an example, the magnet 210 is a permanent magnet. Alternatively, the magnet 210 may be any magnet 210 that may produce a magnetic field 211, such as an electromagnet. As illustrated, a south pole 212 of the magnet 210 is positioned proximal to the trace 108A and a north pole 214 of the magnet 210 is positioned distal to the trace 108A, but alternative configurations are contemplated. The magnet 210 is attached to the substrate layer 216 such as by using surface mount techniques but may, in various examples, be embedded in the substrate 216, or may otherwise be secured with respect to the electronic assembly 100 generally. FIGS. 7A and 7B disclose embodiments whereby the magnet is embedded within substrate 216.

Returning to FIGS. 2A and 2B, the cavity 202 may be formed according to various mechanisms. In an example, the dielectric material 110 is formed in a substantially complete layer and then dielectric material is removed to form the cavity 202. In an example, the dielectric material is removed to form the cavity 202 by using an etching technique. In an example, the etching technique is reactive ion etching, as disclosed in U.S. patent application Ser. No. 13/720,876, filed Dec. 19, 2012, U.S. patent application Ser. No. 14/141,875, filed Dec. 27, 2013, and/or U.S. patent application Ser. No. 13/618,003, filed Sep. 14, 2012. Alternative methods of forming the cavity 202 may be utilized, such as by patterning then developing a photodefinable dielectric material or any other suitable method.

The sensor 102 may operate by actuating the MIP covered trace 108A electromagnetically using a sinusoidal or alternating current generated by the current source 112. The current as generated, in conjunction with the magnetic field 211 created by the magnet 210, may produce a Lorentz force that causes the trace 108A to vibrate within the cavity 202. The current source 112 may be adjusted, such as by sweeping over a frequency range, to output the current so that the frequency substantially matches the mechanical resonant frequency of the trace 108A to produce relatively large lateral displacements of the center 208 of the trace 108A within the cavity 202. The displacement of the center 208 of the trace 108A, in the presence of the magnetic field 211, may produce an induced electromotive force at the trace's 108A resonant frequency. The induced electromotive force may be detected by the frequency detection circuit 114.

When the mass of the trace 108A changes (due to MIP on the beam coupling to analyte), the resonant frequency of the trace 108A may change. The change in the resonant frequency is detectable in the induced electromotive force detected by the frequency detection circuit 114. The change in the resonant frequency of the trace 108A may then be correlated to the analyte concentration in the environment to which the beam is exposed. For example, logic coupled to the sensor may correlate an output signal (indicating the altered resonant frequency) with concentration values found in a look-up table coupled to the logic.

The trace 108A can be actuated with an AC current in the range of few milliamps (e.g., approximately five (5) or fewer milliamps) and consume a minimal amount of power (e.g., approximately ten (10) or fewer mW).

FIGS. 2C-2E are top views of alternative trace geometries, in example embodiments. Such alternative traces 108C, 108D, 108E may include branches 218 near the anchors 200 that produce a different range of resonant frequencies for the traces 108C, 108D, and 108E compared to the straight trace 108A.

FIG. 2F includes a sensor with a cantilever beam in an embodiment of the invention. Note that for the cantilever beam 201, compliant springs 207, 203 on either side of the beam provide a continuous electrical path (from via 213 to via 205) for the actuating current without significantly increasing the stiffness of the cantilever (allowing beam 201 to resonate about via 215). The magnetization direction of the magnet used for actuating the cantilever beam in this case may be different than the magnetization direction of the magnet 210 used to actuate the doubly clamped beam in FIG. 2B.

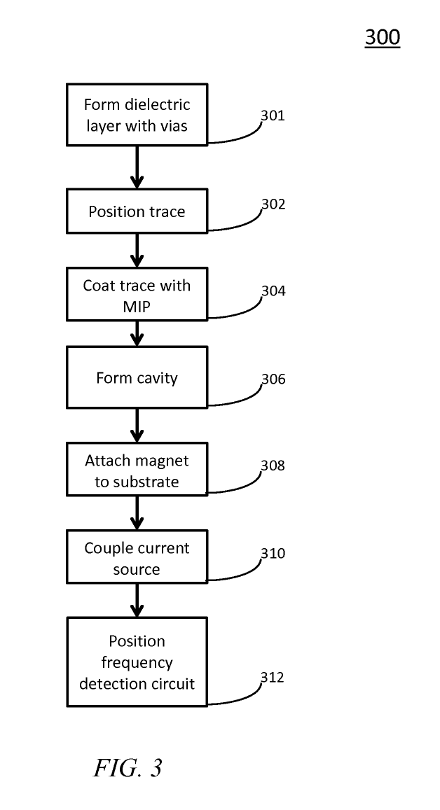

FIG. 3 is a flowchart for a process 300 for making a microelectronic assembly, in another example embodiment.

At operation 301, a dielectric layer with conductive vias is formed. In an example, the dielectric layer is formed by lamination. In an example, the vias are formed by a laser ablation step to create holes in the dielectric layer followed by an electroplating step to plate the vias.

At operation 302, a conductive trace is positioned, at least in part, over the dielectric layer with conductive vias. In an example, the conductive trace may be formed using a lithography process to define the shape of the trace, followed by an electroplating process to create the trace. The conductive trace is substantially mechanically secured to a package layer at a first end by a first anchor and at a second end opposite the first end by a second anchor. In an example, the first and second anchors are connected to the conductive vias in the dielectric layer. In an example, the anchors are portions of the vias.

At operation 304, the conductive trace is coated with a polymer that is then imprinted with an analyte to form a MIP. The analyte may then be removed to functionalize the beam, as described above.

At operation 306, the cavity is formed. In an example, the cavity is formed by removing dielectric material, such as by using reactive ion etching. The conductive trace can now resonate within the cavity at a mass/analyte dependent resonant frequency. The resonance of the trace can be induced through the action of a magnetic field supplied by a magnet and a sinusoidal current supplied by a current source. The sinusoidal current induces a maximal trace displacement, and hence a maximal electromotive force, when a frequency of the sinusoidal current has an approximately equal magnitude to the mass/analyte dependent resonant frequency of the conductive trace. The maximal electromotive force, as induced, has a substantially equal frequency as the mass/analyte dependent resonant frequency of the conductive trace.

At operation 308, a magnet is then attached to the substrate and positioned to induce a magnetic field within the cavity.

At operation 310, the current source is electrically coupled to the conductive trace through at least one of the vias to supply an alternating or sinusoidal current to the trace.

At operation 312, a frequency detection circuit is positioned to detect the frequency of the maximal electromotive force as induced and produce an output proportional to the mass/analyte dependent resonant frequency of the conductive trace. In an example, the frequency detection circuit is a phase-locked loop.

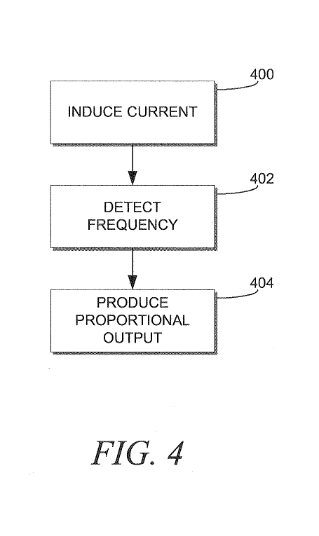

FIG. 4 is a flowchart for using a microelectronic assembly, in an example embodiment. The microelectronic assembly may be the microelectronic assembly 100 or may be another microelectronic assembly.

At operation 400, a current is induced with a current source through the MIP covered conductive trace, the conductive trace being positioned, at least in part, within a cavity in a dielectric material, and a magnet being positioned to induce a magnetic field within the cavity. The conductive trace resonates within the cavity at a mass/analyte dependent resonant frequency through the action of the current and the magnetic field. The sinusoidal current induces a maximal trace displacement, and hence a maximal electromotive force, when a frequency of the sinusoidal current has an approximately equal magnitude to the mass/analyte dependent resonant frequency of the conductive trace. The maximal electromotive force, as induced, has a substantially equal frequency as the mass/analyte dependent resonant frequency of the conductive trace.

In an example, the conductive trace is substantially mechanically secured to a package layer at a first end by a first anchor and at a second end opposite the first end by a second anchor. In an example, the first and second anchors are positioned to allow the conductive trace to move laterally as the conductive trace resonates. In an example, the first and second anchors are connected to vias while in other embodiments they are portions of the vias. In an example, the current source is electrically coupled to the conductive trace through at least one of the vias. In an example, the conductive trace is comprised, at least in part, of copper.

At operation 402, the frequency of the electromotive force as induced is detected with a frequency detection circuit. In an example, the frequency detection circuit is a phase-locked loop.

At operation 404, an output proportional to the mass/analyte dependent resonant frequency of the conductive trace is produced with the frequency detection circuit.

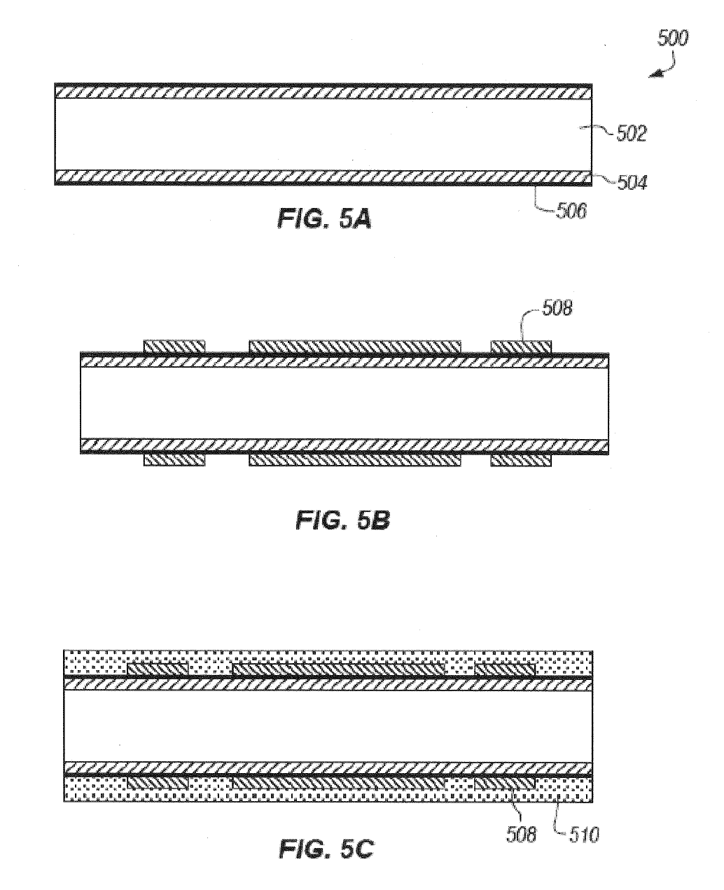

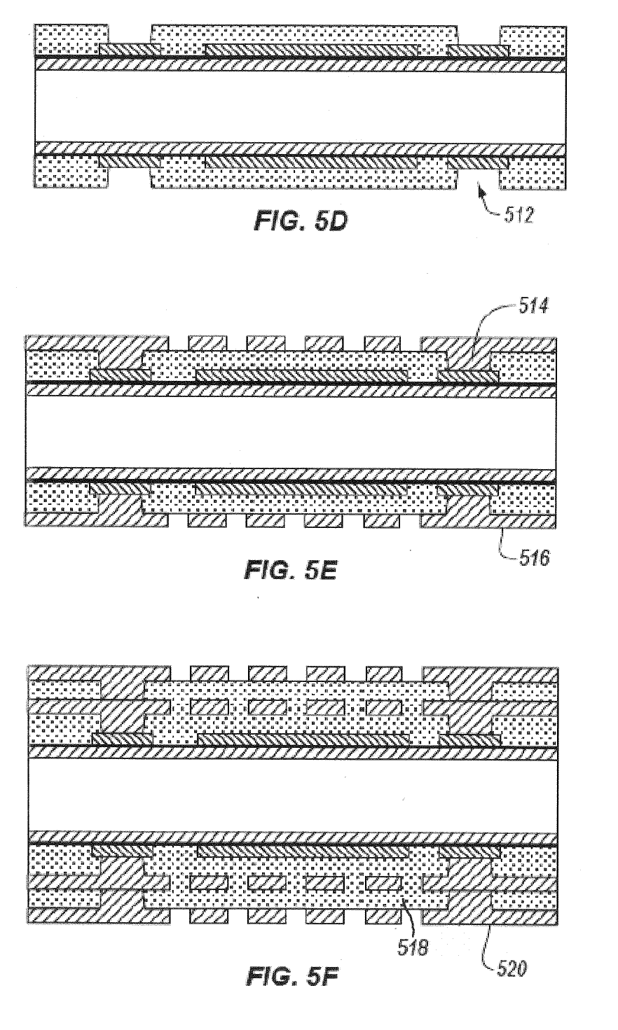

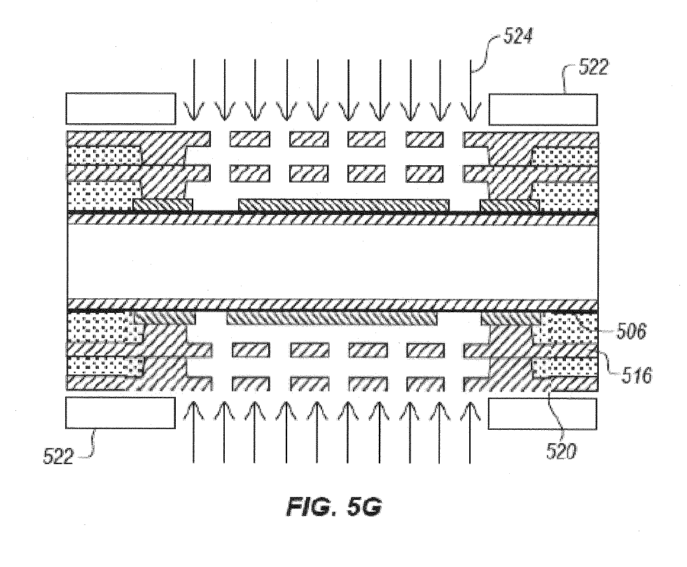

FIG. 5A is a side cross-sectional diagram that shows an example of the use of an incoming peelable core 500 for use in fabricating a chemical analyte sensor in a package. The sensor may be fabricated using certain substrate processing techniques. The incoming peelable core has an organic carrier 502 at its center which is covered on both sides with a laminated copper foil 504 and a peelable copper layer 506. The copper layer 506 is weakly adhered to the laminated copper foil 504 so that the copper layer 506 can be peeled off after all the substrate fabrication processes are completed.

In FIG. 5B a dry film resist (DFR) pattern is used to apply copper plating according to a specific intended pattern. A pattern of lands 508 for routing layers and connections are formed on both sides of the core over the peelable Copper layer 506. In FIG. 5C a buildup layer 510 is laminated over the copper plating. In FIG. 5D laser etching is used to form valleys 512 in the buildup lamination. In FIG. 5E copper is applied into the valleys 512 to form vias 514 and a first metal layer 516 is applied over the buildup. The first metal layer 516 may contain the traces comprising the chemical analyte sensor beam (or beams) and may also include routing layers as desired to connect the sensor with the vias and certain other components that are to be formed. In an embodiment, the beam may then be coated with a MIP to functionalize its surface and make it selective to the analyte that is to be sensed.

In FIG. 5F the operations of depositing buildup and patterning metal over the buildup are repeated with a second layer of dielectric 518 and a second metal layer 520 to form a mesh pattern over the dielectric and over the first metal layer 516.

In FIG. 5G a plasma mask 522 is applied on both sides of the structure and buildup etching 524 is applied to the structure. The mask may be a hard mask that is patterned on top of second metal layer 520 and then removed after plasma etching. The mask determines which areas will be etched and the buildup in the exposed area is completely removed. This may provide for two metal layers 516, 520 with no or substantially no intervening materials between them and the location where the sensor is located. However, dielectrics remain in areas that were not exposed to the etching process.

Further processing (not shown) may then occur. For example, additional metal layers may be employed to form C4 bumps and the like and couple external components to the first metal layer 516 and second metal layer 520. Alternatively any of a variety of other electrical technologies may be used to connect external components depending on the particular implementation. Temporary protection may be placed over the exposed beam to ensure further processing does not harm the beam. The temporary protection (e.g., solder resist) may later be removed to ensure the beam is exposed to future chemical analytes. Eventually the peelable copper in the core may be removed to separate the top and bottom substrate portions on either side of the core. Further, a magnet may be placed opposite the void that exposes the beam to the atmosphere. The magnet may be located where the peelable copper was removed (i.e., on the “bottom” of the device so the magnet's field still encompasses the beam but the magnet itself does not block the introduction of analyte to the MIP covered beam).

FIGS. 7A and 7B are top and side views of a sensor, respectively, in an embodiment of the invention. These figures are similar to FIGS. 2A and 2B but depict embodiments whereby the magnet 210 is embedded within substrate 216 instead of being attached to the substrate as shown in FIG. 2B. For brevity, like components have retained their identifiers from FIGS. 2A and 2B (e.g., the substrate is labeled as element 216 in FIGS. 2B and 7B) and are not described again. The embodiments of FIGS. 2C, 2D, 2E, and 2F may be similarly modified to provide a magnet that is embedded in the substrate instead of attached to the substrate.

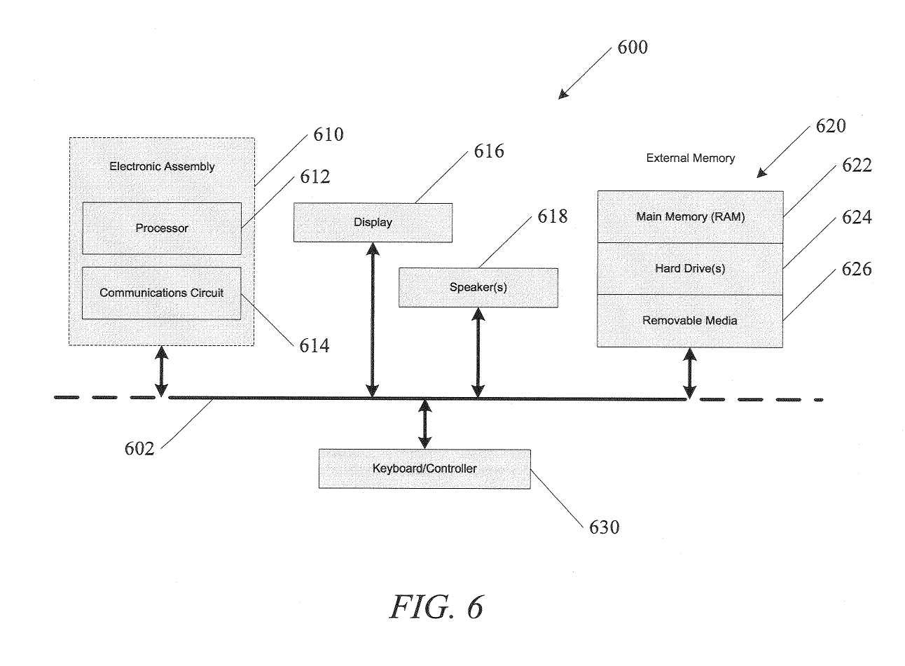

An example of an electronic device using electronic assemblies as described in the present disclosure is included to show an example of a higher level device application for the disclosed subject matter. FIG. 6 is a block diagram of an electronic device 600 incorporating at least one electronic assembly, such as an electronic assembly 100 or other electronic or microelectronic assembly related to examples herein. The electronic device 600 is merely one example of an electronic system in which embodiments of the present invention can be used. Examples of electronic devices 600 include, but are not limited to personal computers, tablet computers, mobile telephones, personal data assistants, MP3 or other digital music players, wearable devices, Internet of things (IOTS) devices, etc. In this example, the electronic device 600 comprises a data processing system that includes a system bus 602 to couple the various components of the system. The system bus 602 provides communications links among the various components of the electronic device 600 and can be implemented as a single bus, as a combination of busses, or in any other suitable manner.

An electronic assembly 610 is coupled to the system bus 602. The electronic assembly 610 can include any circuit or combination of circuits. In one embodiment, the electronic assembly 610 includes a processor 612 which can be of any type. As used herein, “processor” means any type of computational circuit, such as but not limited to a microprocessor, a microcontroller, a complex instruction set computing (CISC) microprocessor, a reduced instruction set computing (RISC) microprocessor, a very long instruction word (VLIW) microprocessor, a graphics processor, a digital signal processor (DSP), multiple core processor, or any other type of processor or processing circuit.

Other types of circuits that can be included in the electronic assembly 610 are a custom circuit, an application-specific integrated circuit (ASIC), or the like, such as, for example, one or more circuits (such as a communications circuit 614) for use in wireless devices like mobile telephones, pagers, personal data assistants, portable computers, two-way radios, and similar electronic systems. The IC can perform any other type of function.

The electronic device 600 can also include an external memory 620, which in turn can include one or more memory elements suitable to the particular application, such as a main memory 622 in the form of random access memory (RAM), one or more hard drives 624, and/or one or more drives that handle removable media 626 such as compact disks (CD), digital video disk (DVD), and the like.

The electronic device 600 can also include a display device 616, one or more speakers 618, and a keyboard and/or controller 630, which can include a mouse, track connection, touch screen, voice-recognition device, or any other device that permits a system user to input information into and receive information from the electronic device 600.

Bus 602 may further couple to input/output ports such as ports 106 of FIG. 1 or traces 108, either of which may further couple a sensor package including embodiments of the sensors described herein.

Processor 612 may couple to logic to analyze data and provide actionable feedbacks to users. That logic may be included on a substrate (e.g., the same substrate the sensor is on) or coupled thereto. The logic may take into account other factors besides those directly sensed. For example, in fitness usage the level of acetone or ammonia may not necessarily represent the body chemical or physiological conditions because they can be produced in high levels due to protein rich (ammonia indicator) or fat-rich (acetone indicator) diets. When analyzing the data, other factors (e.g., diet) may be taken into consideration.

The logic may include program instructions used to cause a general-purpose or special-purpose processing system that is programmed with the instructions to perform the operations described herein. Alternatively, the operations may be performed by specific hardware components that contain hardwired logic for performing the operations, or by any combination of programmed computer components and custom hardware components. The methods described herein (e.g., determining a concentration of a detected analyte) may be provided as (a) a computer program product that may include one or more machine readable media having stored thereon instructions that may be used to program a processing system or other electronic device to perform the methods or (b) at least one storage medium having instructions stored thereon for causing a system to perform the methods. The term “machine readable medium” or “storage medium” used herein shall include any medium that is capable of storing or encoding a sequence of instructions (transitory media, including signals, or non-transitory media) for execution by the machine and that cause the machine to perform any one of the methods described herein. The term “machine readable medium” or “storage medium” shall accordingly include, but not be limited to, memories such as solid-state memories, optical and magnetic disks, read-only memory (ROM), programmable ROM (PROM), erasable PROM (EPROM), electrically EPROM (EEPROM), a disk drive, a floppy disk, a compact disk ROM (CD-ROM), a digital versatile disk (DVD), flash memory, a magneto-optical disk, as well as more exotic mediums such as machine-accessible biological state preserving or signal preserving storage. A medium may include any mechanism for storing, transmitting, or receiving information in a form readable by a machine, and the medium may include a medium through which the program code may pass, such as antennas, optical fibers, communications interfaces, etc. Program code may be transmitted in the form of packets, serial data, parallel data, etc., and may be used in a compressed or encrypted format. Furthermore, it is common in the art to speak of software, in one form or another (e.g., program, procedure, process, application, module, logic, and so on) as taking an action or causing a result. Such expressions are merely a shorthand way of stating that the execution of the software by a processing system causes the processor to perform an action or produce a result.

A module as used herein refers to any hardware, software, firmware, or a combination thereof. Often module boundaries that are illustrated as separate commonly vary and potentially overlap. For example, a first and a second module may share hardware, software, firmware, or a combination thereof, while potentially retaining some independent hardware, software, or firmware. In one embodiment, use of the term logic includes hardware, such as transistors, registers, or other hardware, such as programmable logic devices. However, in another embodiment, logic also includes software or code integrated with hardware, such as firmware or micro-code.

As used herein, “analyte”, “biomarker”, and “target molecule” refer to molecules to be analyzed, detected, or sensed. Molecules are at times referred to as “biomarkers” when they originate from a biological system and are a form of “analyte”. A “volatile organic compound” (VOC) is a subclass of organic compounds and can be present in gas or liquid form and is a form of analyte. As used herein, “package” is the housing of a chip or discrete device and electrically interconnects the chip with outside circuitry. The package also provides physical and chemical protection of the chip and is designed to dissipate heat generated by the chip.

The following examples pertain to further embodiments.

Example 1 includes an electronic package, comprising: a cavity formed within a dielectric material; a beam located in the cavity and having a long axis in a horizontal plane; an interconnect to couple the beam to a current source; a magnet coupled to the cavity; and a polymer, on the beam, having an affinity to a chemical analyte; wherein (a) a vertical axis intersects the magnet, the cavity, and the beam; (b) in a first state the beam and the polymer, which is not coupled to the chemical analyte, collectively have a first mass and resonate at a first resonant frequency when the beam conducts a first current from the current source; (c) in a second state the beam and the polymer, which is coupled to the chemical analyte, collectively have a second mass that is greater than the first mass and resonate at a second resonant frequency, unequal to the first resonant frequency, when the beam conducts a second current from the current source.

The MIP may include a monolayer of monomers coupled to an oxide (e.g., covalently) and then “templated” or “programmed” with analytes to form the MIP. A monolayer polymer initiator may be used to control polymer thickness over the beam. After the analytes are removed, the MIP is produced. Portions of the MIP may be covered with a reversible protection layer (e.g., photoresist, oxide), which may be removed in areas to provide windows such that analyte may be given a chance to interact with the MIP for sensing.

As used herein, “having an affinity to a chemical analyte” includes having a specificity to an analyte which is used to sense the analyte (e.g., a MIP has an affinity to the analyte the MIP was programmed (e.g., imprinted) with). Of course saying the polymer has an affinity to one analyte does not preclude the same polymer from having an affinity for second, third and fourth analytes as well.

In an embodiment the polymer does not couple to beam directly but does so indirectly through an oxide that is on the beam. The oxide is modified first with silane, phosphonate or other attachment chemistry. The modifying molecule may terminate with a functional group selected from the group comprising amines, carboxyls, aldehydes, thiols, hydroxyls, and epoxies. In an embodiment the polymer couples to the oxide layer via a member selected from the group comprising amines, carboxyls, aldehydes, thiols, hydroxyls, and epoxies.

As used herein, “programming” a polymer connotes instilling an affinity to a chemical analyte (e.g., imprinting a polymer to create a MIP). For example, a manufacturer may ship a polymer covered beam without the polymer having been programmed. The manufacturer's customer may instead program the polymer at a later time. Some embodiments may allow a manufacturer to ship a beam not yet coated with the polymer such that a customer may coat the beam and program the polymer.

While embodiments of the beams discussed herein have primarily been linear beams, other embodiments are not limited to a linear shape and the beams may be curved, have voids within the beams, and the like.

While most embodiments have included a magnet, other embodiments may allow for the sensor of example 1 to ship to customers without the magnet attached so that the customer can instead supply his or her own magnet at a later time.

In the above example, the first and second currents may be unequal to each other (e.g., may have different frequencies but both be sinusoidal).

In the above example, coupling the analyte to the polymer includes a molecule or chemical component form fitting to a molecular imprint of the polymer, thereby adding mass to the beam and changing the resonant frequency of the beam.

In example 2 the subject matter of the Example 1 can optionally include frequency detection logic to detect the second frequency.

This logic may be included within a module that includes software, hardware, and/or firmware to detect frequency.

In example 3 the subject matter of the Examples 1-2 can optionally include wherein the frequency detection logic includes at least one of a phase locked loop (PLL) circuit, an analog/digital converter with a digital block, or any open or closed loop method for estimating frequency.

In example 4 the subject matter of the Examples 1-3 can optionally include chemical analyte logic to produce a signal proportional to an amount of chemical analyte coupled to the polymer in the second state.

For example, the frequency of the induced electromotive force at the beam's resonant frequency may be detected. The logic may then correlate this frequency to a frequency located in a look up table. The frequency in the look up table may correspond to a certain concentration of an analyte, which may be communicated to a user. The communication may be visual (via a display), auditory (via a speaker), and/or tactile (via a vibrating member within a smartwatch).

In example 5 the subject matter of the Examples 1-4 can optionally include wherein the chemical analyte logic comprises a look-up table that associates a plurality of chemical analyte concentrations that correspond to a plurality of resonant frequencies.

In example 6 the subject matter of the Examples 1-5 can optionally include wherein the polymer has the affinity to the analyte when the polymer includes a member selected from the group comprising: a molecular imprint specific to the analyte, a physical printing specific to the analyte, and a photolithographical printing specific to the analyte.

In example 7 the subject matter of the Examples 1-6 can optionally include wherein a middle portion of the beam includes a vertical cross-section, orthogonal to the long axis that is completely surrounded by a void.

In such a case the middle portion of the beam (and the vertical cross-section) may have 360 degrees of freedom with no dielectric above, below or on either side of the beam (while still having other beam portions distal and proximal to the middle portion in relation to one end of the beam). This may include cantilever beam embodiments. Thus, the middle portion of the beam is completely surrounded by a void on all sides except where it connects to the rest of the beam. In another version of example 7 the subject matter of the Examples 1-6 can optionally include embodiments in which the beam is clamped on both ends. Other embodiments may provide that the beam is clamped on only one end with the other end being free to move (cantilever beam).

In another version of example 7 the subject matter of the Examples 1-6 can optionally include wherein a middle portion of the beam, located between first and second side portions of the beam, is completely surrounded by a void except for connections to the first and second side portions of the beam.

In example 8 the subject matter of the Examples 1-7 can optionally include wherein the void is in fluid communication with the cavity and with an exterior outlet of the package, the exterior outlet being coupled to atmospheric conditions.

For example, FIG. 5G shows the removal of dielectric material to ensure this fluid communication occurs. The “fluid” here includes gaseous and liquid phases of chemical analyte. In such an embodiment, the beam is also in fluid communication with the exterior outlet.

In example 9 the subject matter of the Examples 1-8 can optionally include wherein the analyte is selected from the group comprising liquid ketones, liquid alcohols, liquid aldehydes, volatile organic compounds (VOCs), metal ions, biomarkers, hormones, liquid esters, carboxylic acids, ethers, amines, halohydrocarbons (with F, Cl, Br, or I), proteins, and polypeptides.

VOCs may include, without limitation, Chloromethane, Bromomethane, Vinyl chloride, Chloroethane, Methylene chloride, Acetone, Carbon disulfide, 1,1-Dichloroethene, 1,1-Dichloroethane, Total-1,2-dichloroethene, Chloroform, 1,2-Dichloroethane, 2-Butanone, 1,1,1-Trichloroethane, Carbon tetrachloride, Vinyl acetate, Bromodichloromethane, 1,2-Dichloropropane, Cis-1,3-dichloropropene, Trichloroethene, Dibromochloromethane, 1,1,2-Trichloroethane, Benzene, Trans-1,3-dichloropropene, Bromoform, 4-Methyl-2-pentanone, 2-Hexanone, Tetrachloroethene, 1,1,2,2-Tetrachloroethane, Toluene, Chlorobenzene, Ethylbenzene, Styrene, and Total Xylenes.

Analytes may be in a gaseous phase, including the above VOCs and/or other VOCs from farms, industries, a person's breath or skin, and the like. The above mentioned metal ions may include, for example, K+, Na+, Mg++, Hg+, and the like. Analytes may further include small organic molecules (e.g., bisphenolic A, antibiotics, depressants, herbicides, and the like), biomarkers (e.g., troponin, c-reactive proteins, IL-6, IgE, and the like), and steroids and/or other hormones. Analytes in liquid phase may be included in water, a soil extract, a food extract, blood, urine, saliva, and other bodily fluids.

Analytes may also include liquid esters, carboxylic acids, ethers, amines, halohydrocarbons (e.g., including F, Cl, Br, and/or I). Biomarkers may include small molecules, proteins, carbohydrates, nucleic acids, and/or lipids. Hormones may include vitamins, proteins and/or polypeptides.

In example 10 the subject matter of the Examples 1-9 can optionally include wherein the polymer includes a member selected from the group comprising peptides and aptamers.

For embodiments that sense analytes in liquid, aptamers may be used. Aptamers are highly selective polymers for recognizing a wide variety of analytes types such as bacteria, cells, viruses, proteins, nucleotide sequences, heavy metals, organic and inorganic compounds for environmental and health related sensing applications. Specifically, aptamers may be oligonucleotide or peptide molecules that bind to a specific target molecule. Since aptamers are artificial nucleic acid ligands they can be designed for target analytes and generated by in vitro selection through partition and amplification. Aptamers are structurally versatile because they have basic stem-loop arrangements that form proper three-dimensional structures. These structures facilitate the formation of a complex with the target molecule to influence the target's function. Aptamers have high affinities to their targets, with dissociation constants at the low-picomolar (pM) level, comparable to or better than antibodies, including better stability, no batch variation, smaller sizes, and easier modification. Aptamers can be implemented as reusable sensing elements. Other embodiments use still other forms of chemical interface, such as fluorine-containing polymers (F-polymer).

In example 11 the subject matter of the Examples 1-10 can optionally include wherein the polymer is reusable and does not degrade in response to coupling to the analyte.

In example 12 the subject matter of the Examples 1-11 can optionally include an additional beam having an additional long axis, parallel to the long axis, in the horizontal plane; an additional interconnect to couple the additional beam to at least one of the current source and an additional current source; an additional polymer, on the additional beam, having an additional affinity to an additional chemical analyte that is different from the chemical analyte; wherein (a) an additional vertical axis intersects the magnet and the additional beam; (b) in an additional first state the additional beam and the additional polymer, which is not coupled to the additional chemical analyte, collectively have an additional first mass and resonate at an additional first resonant frequency when the additional beam conducts an additional first current from the at least one of the current source and the additional current source; and (c) in an additional second state the additional beam and the additional polymer, which is coupled to the additional chemical analyte, collectively have an additional second mass that is greater than the additional first mass and resonate at an additional second resonant frequency unequal to the additional first resonant frequency when the additional beam conducts an additional second current from the at least one of the current source and the additional current source.

An embodiment uses sensors (all in a single package) to ensure specificity and multiplexing detection of chemical analytes. The sensors are modified with different polymers that are either pre-synthesized beforehand or in-situ synthesized. For example, a manufacturer may ship sensors before the molecular imprinting takes place (leaving the imprinting step to the customer). An embodiment achieves site-selective modification on a beam via inkjet-printing. Another embodiment achieves site-selective modification on a beam via screen printing. Other embodiments use a photoresist patterning process, in which given sites are accessible to the reagent (coating chemicals) while other sites not to be modified are protected by a photoresist. The protection and stripping steps can be repeated for multiple site surface modifications. This can be done on singulated die or at wafer level. Furthermore, multiple steps on the same site can be performed to synthesize desired chemical polymers in situ. Use of a photolithography process generates small features such that different features (e.g., different chemical contents) can be made within a small space (<100 um). Also, the shape of the spot may have straight boundary lines as opposed to printing. See also U.S. patent application Ser. No. 14/669,514, assigned to Intel Corporation of Santa Clara, Calif., USA.

Example 12 may include a single package with two different beams that focus on two different analytes. The beams may share a current source (which couples to the beams through a multiplexor (MUX) that toggles current to each beam) or have different current sources. The example may allow for simultaneous sensing of two different analytes.

In example 13 the subject matter of the Examples 1-12 can optionally include an additional beam having an additional long axis, parallel to the long axis, in the horizontal plane; an additional interconnect to couple the additional beam to at least one of the current source and an additional current source; wherein (a) the polymer is on the additional beam; (b) an additional vertical axis intersects the magnet and the additional beam; (c) in an additional first state the additional beam and the polymer, which is not coupled to the chemical analyte, collectively have an additional first mass and resonate at an additional first resonant frequency when the additional beam conducts an additional first current from the at least one of the current source and the additional current source; and (c) in an additional second state the additional beam and the polymer, which is coupled to the chemical analyte, collectively have an additional second mass that is greater than the additional first mass and resonate at an additional second resonant frequency when the additional beam conducts an additional second current from the at least one of the current source and the additional current source.

Example 13 may include a single package with two different beams that focus on the same analyte. The beams may share a current source (which couples to the beams through a MUX that toggles current to each beam) or have different current sources. The example may allow for simultaneous sensing of the same analyte, thereby allowing for averaging and the like and providing a better signal to noise ratio (SNR). In other words, the quality of the sensing may increase.

In example 14 the subject matter of the Examples 1-13 can optionally include wherein (a) the polymer has an additional affinity to an additional chemical analyte that is different from the chemical analyte; (b) in an additional first state the beam and the polymer, which is not coupled to the additional chemical analyte, collectively have an additional first mass and resonate at an additional first resonant frequency when the beam conducts an additional first current from the current source; and (c) in an additional second state the beam and the polymer, which is coupled to the additional chemical analyte, collectively have an additional second mass that is greater than the additional first mass and resonate at an additional second resonant frequency unequal to the additional first resonant frequency when the beam conducts an additional second current from the current source.

In another version of example 14 the subject matter of the Examples 1-13 can optionally include wherein (a) the polymer has an additional affinity to an additional chemical analyte that is different from the chemical analyte; and (b) in an additional second state the beam and the polymer, which is coupled to the additional chemical analyte, collectively have an additional second mass that is greater than the first mass and resonate at an additional second resonant frequency unequal to the first resonant frequency when the beam conducts an additional second current from the current source.

Thus, in this example a single beam can have two different molecular imprints. Resonant frequencies may be known for when one of the analytes is detected, when the other analyte is detected, and when both of the analytes are detected—all with values in a look-up table to illuminate what is being sensed.

In example 15 the subject matter of the Examples 1-14 can optionally include a control beam having an additional long axis, parallel to the long axis, in the horizontal plane; an additional interconnect to couple the control beam to at least one of the current source and an additional current source; a control polymer, on the control beam, having no molecular imprinting and no affinity to the chemical analyte or any other chemical analyte; wherein (a) an additional vertical axis intersects the magnet and the control beam; and (b) in an additional first state the control beam and the control polymer, which is not coupled to the chemical analyte, collectively have an additional first mass and resonate at an additional first resonant frequency that serves as a control to the beam and the polymer when the control beam conducts an additional first current from the at least one of the current source and the additional current source.

For example, an embodiment may use a “differential measurement” for sensing. For example, two sensor beams may be placed adjacent each other. One of the sensors may have an analyte specific capture polymer and the other may not (i.e., has no molecular imprint). When both are exposed to a sample they will respond to physical and chemical changes (e.g., change in mass). However, there will be a difference between the two beam's reactions (e.g., a resonant frequency for one of the beams) and the difference is caused by the analyte being sensed by the beam sensor with the analyte specific capture polymer.

In another version of example 15 the subject matter of the Examples 1-14 can optionally include a control beam having an additional long axis, parallel to the long axis, in the horizontal plane; an additional interconnect to couple the control beam to at least one of the current source and an additional current source; wherein (a) an additional vertical axis intersects the magnet and the control beam; and (b) in an additional first state the control beam, which is not coupled to the chemical analyte, has an additional first mass and resonates at an additional first resonant frequency that serves as a control to the beam and the polymer when the control beam conducts an additional first current from the at least one of the current source and the additional current source.

Thus, in this example the control beam may not have a polymer coating (but in other embodiments it may). As a result, the control beam should have a fairly consistent resonant frequency even upon exposure to the analyte being sensed. This can allow differential measurements (e.g., using a comparator) to be conducted between the control beam and the sensing beam.

In example 16 the subject matter of the Examples 1-15 can optionally include wherein the beam is included in a metal layer that extends from the beam to a logic portion of a system on a chip (SoC) that comprises a processor.

In example 17 the subject matter of the Examples 1-16 can optionally include wherein the beam is a cantilever beam.

In example 18 the subject matter of the Examples 1-17 can optionally include wherein the interconnect mechanically anchors the beam to a layer in the package while still allowing a portion of the beam to deflect when the beam resonates at the first resonant frequency.

In example 19 the subject matter of the Examples 1-18 can optionally include wherein the interconnect is a via and the layer is a metal layer.

In another version of example 19 the subject matter of the Examples 1-18 can optionally include wherein the interconnect is a via, the layer is a metal layer, and the polymer is a molecular imprint polymer (MIP).

In example 20 the subject matter of the Examples 1-19 can optionally include the current source, wherein the current source is electrically coupled to the beam through the via.

Example 21 includes a method comprising: forming a metal layer; forming a conductive trace, from the metal layer, that forms a beam having a long axis in a horizontal plane; forming an interconnect to couple the beam to a current source; coupling a polymer to the beam, the polymer having an affinity to a chemical analyte; forming a dielectric layer on a substrate and above, below, and on each side of the beam; removing a portion of the dielectric layer to form a cavity that includes the beam; coupling a magnet to the metal layer; wherein (a) a vertical axis intersects the magnet, the cavity, and the beam; (b) in a first state the beam and the polymer, which is not coupled to the chemical analyte, collectively have a first mass and resonate at a first resonant frequency when the beam conducts a first current from the current source; (c) in a second state the beam and the polymer, which is coupled to the chemical analyte, collectively have a second mass that is greater than the first mass and resonate at a second resonant frequency, unequal to the first resonant frequency, when the beam conducts a second current from the current source.

Please note the order of operations in the above example may be rearranged in varying embodiments.

Another version of Example 21 includes a method comprising: forming a metal layer; forming a conductive trace, from the metal layer, that forms a beam having a long axis in a horizontal plane; forming interconnect to couple the beam to a current source; forming a dielectric layer on a substrate and above, below, and on each side of the beam; removing a portion of the dielectric layer to form a cavity that includes the beam; coupling a magnet to the metal layer, coupling a polymer to the beam, the polymer having an affinity to a chemical analyte; wherein (a) a vertical axis intersects the magnet, the cavity, and the beam; (b) in a first state the beam and the polymer, which is not coupled to the chemical analyte, collectively have a first mass and resonate at a first resonant frequency when the beam conducts a first current from the current source; (c) in a second state the beam and the polymer, which is coupled to the chemical analyte, collectively have a second mass that is greater than the first mass and resonate at a second resonant frequency, unequal to the first resonant frequency, when the beam conducts a second current from the current source.

In example 22 the subject matter of the Example 21 can optionally include including the current source in a package that also includes the beam.

Example 23 includes a system comprising: a cavity formed within an insulating material; a beam located in the cavity and having a long axis in a horizontal plane; an interconnect to couple the beam to a current source; a magnet coupled to the cavity; and a polymer on the beam; wherein (a) a vertical axis intersects the magnet, the cavity, and the beam; (b) when in a first state the beam and the polymer, which is not coupled to a chemical analyte, collectively have a first mass and resonate at a first resonant frequency when the beam conducts a first current from the current source; and (c) when in a second state the beam and the polymer, which is coupled to the chemical analyte, collectively have a second mass that is greater than the first mass and resonate at a second resonant frequency, unequal to the first resonant frequency, when the beam conducts a second current from the current source.

In example 24 the subject matter of Example 23 can optionally include wherein the polymer is programmed to have an affinity to the chemical analyte.

Thus, example 23 may have a polymer that has not yet been programmed (but eventually will be by a, for example, downstream customer).

In example 25 the subject matter of Examples 23-24 can optionally include a logic module (e.g., a programmed field programmable gate array) to produce a signal proportional to an amount of the chemical analyte coupled to the polymer in the second state.

The foregoing description of the embodiments of the invention has been presented for the purposes of illustration and description. It is not intended to be exhaustive or to limit the invention to the precise forms disclosed. This description and the claims following include terms, such as left, right, top, bottom, over, under, upper, lower, first, second, etc. that are used for descriptive purposes only and are not to be construed as limiting. For example, terms designating relative vertical position refer to a situation where a device side (or active surface) of a substrate or integrated circuit is the “top” surface of that substrate; the substrate may actually be in any orientation so that a “top” side of a substrate may be lower than the “bottom” side in a standard terrestrial frame of reference and still fall within the meaning of the term “top.” The term “on” as used herein (including in the claims) does not indicate that a first layer “on” a second layer is directly on and in immediate contact with the second layer unless such is specifically stated; there may be a third layer or other structure between the first layer and the second layer on the first layer. The embodiments of a device or article described herein can be manufactured, used, or shipped in a number of positions and orientations. Persons skilled in the relevant art can appreciate that many modifications and variations are possible in light of the above teaching. Persons skilled in the art will recognize various equivalent combinations and substitutions for various components shown in the Figures. It is therefore intended that the scope of the invention be limited not by this detailed description, but rather by the claims appended hereto.

What is claimed is:

1. An electronic package, comprising:
a cavity formed within a dielectric material;
a beam located in the cavity and having a long axis in a horizontal plane;
an interconnect to couple the beam to a current source;
a magnet coupled to the cavity; and
a polymer, on the beam, having an affinity to a chemical analyte;
wherein (a) a vertical axis intersects the magnet, the cavity, and the beam; (b) in a first state the beam and the polymer, which is not coupled to the chemical analyte, collectively have a first mass and resonate at a first resonant frequency when the beam conducts a first current from the current source; and (c) in a second state the beam and the polymer, which is coupled to the chemical analyte, collectively have a second mass that is greater than the first mass and resonate at a second resonant frequency, unequal to the first resonant frequency, when the beam conducts a second current from the current source.

2. The package of claim 1 comprising frequency detection logic to detect the second frequency.

3. The package of claim 2 wherein the frequency detection logic includes at least one of a phase lock loop (PLL) circuit and an analog to digital converter.

4. The package of claim 2 comprising chemical analyte logic to produce a signal proportional to an amount of chemical analyte coupled to the polymer in the second state.

5. The package of claim 4, wherein the chemical analyte logic comprises a memory storing a look-up table that associates a plurality of chemical analyte concentrations that correspond to a plurality of resonant frequencies.

6. The package of claim 1, wherein the polymer has the affinity to the analyte when the polymer includes a member selected from the group comprising: a molecular imprint specific to the analyte, a physical printing specific to the analyte, and a photolithographical printing specific to the analyte.

7. The package of claim 1, wherein a middle portion of the beam, located between first and second side portions of the beam, is completely surrounded by a void except for connections to the first and second side portions of the beam.

8. The package of claim 7, wherein the void is in fluid communication with the cavity and with an exterior outlet of the package, the exterior outlet being coupled to atmospheric conditions.

9. The package of claim 1, wherein the analyte is selected from the group comprising liquid ketones, liquid alcohols, liquid aldehydes, volatile organic compounds (VOCs), metal ions, biomarkers, hormones, liquid esters, carboxylic acids, ethers, amines, halohydrocarbons (with F, Cl, Br, or I), proteins, and polypeptides.

10. The package of claim 9, wherein the polymer includes a member selected from the group comprising peptides and aptamers.

11. The package of claim 1, wherein the polymer is reusable and does not degrade in response to coupling to the analyte.

12. The package of claim 1 comprising:
an additional beam having an additional long axis, parallel to the long axis, in the horizontal plane;
an additional interconnect to couple the additional beam to at least one of the current source and an additional current source;
an additional polymer, on the additional beam, having an additional affinity to an additional chemical analyte that is different from the chemical analyte;
wherein (a) an additional vertical axis intersects the magnet and the additional beam; (b) in an additional first state the additional beam and the additional polymer, which is not coupled to the additional chemical analyte, collectively have an additional first mass and resonate at an additional first resonant frequency when the additional beam conducts an additional first current from the at least one of the current source and the additional current source; and (c) in an additional second state the additional beam and the additional polymer, which is coupled to the additional chemical analyte, collectively have an additional second mass that is greater than the additional first mass and resonate at an additional second resonant frequency unequal to the additional first resonant frequency when the additional beam conducts an additional second current from the at least one of the current source and the additional current source.

13. The package of claim 1 comprising:
an additional beam having an additional long axis, parallel to the long axis, in the horizontal plane;
an additional interconnect to couple the additional beam to at least one of the current source and an additional current source;
wherein (a) the polymer is on the additional beam; (b) an additional vertical axis intersects the magnet and the additional beam; (c) in an additional first state the additional beam and the polymer, which is not coupled to the chemical analyte, collectively have an additional first mass and resonate at an additional first resonant frequency when the additional beam conducts an additional first current from the at least one of the current source and the additional current source; and (c) in an additional second state the additional beam and the polymer, which is coupled to the chemical analyte, collectively have an additional second mass that is greater than the additional first mass and resonate at an additional second resonant frequency when the additional beam conducts an additional second current from the at least one of the current source and the additional current source.

14. The package of claim 1, wherein (a) the polymer has an additional affinity to an additional chemical analyte that is different from the chemical analyte; and (b) in an additional second state the beam and the polymer, which is coupled to the additional chemical analyte, collectively have an additional second mass that is greater than the first mass and resonate at an additional second resonant frequency unequal to the first resonant frequency when the beam conducts an additional second current from the current source.

15. The package of claim 1 comprising:
a control beam having an additional long axis, parallel to the long axis, in the horizontal plane; and
an additional interconnect to couple the control beam to at least one of the current source and an additional current source;
wherein (a) an additional vertical axis intersects the magnet and the control beam; and (b) in an additional first state the control beam, which is not coupled to the chemical analyte, has an additional first mass and resonates at an additional first resonant frequency that serves as a control to the beam and the polymer when the control beam conducts an additional first current from the at least one of the current source and the additional current source.

16. The package of claim 1 wherein the beam is included in a metal layer that extends from the beam to a logic portion of a system on a chip (SoC) that comprises a processor.

17. The package of claim 1, wherein the beam is a cantilever beam.

18. The package of claim 1, wherein the interconnect mechanically anchors the beam to a layer in the package while still allowing a portion of the beam to deflect when the beam resonates at the first resonant frequency.

19. The package of claim 18, wherein the interconnect is a via, the layer is a metal layer, and the polymer is a molecular imprint polymer (MIP).

20. The package of claim 19 further comprising the current source, wherein the current source is electrically coupled to the beam through the via.

21. A method comprising:
forming a metal layer;
forming a conductive trace, from the metal layer, that forms a beam having a long axis in a horizontal plane;
forming an interconnect to couple the beam to a current source;
coupling a polymer to the beam, the polymer having an affinity to a chemical analyte;
forming a dielectric layer on a substrate and above, below, and on each side of the beam;
removing a portion of the dielectric layer to form a cavity that includes the beam;
coupling a magnet to the metal layer,
wherein (a) a vertical axis intersects the magnet, the cavity, and the beam; (b) in a first state the beam and the polymer, which is not coupled to the chemical analyte, collectively have a first mass and resonate at a first resonant frequency when the beam conducts a first current from the current source; (c) in a second state the beam and the polymer, which is coupled to the chemical analyte, collectively have a second mass that is greater than the first mass and resonate at a second resonant frequency, unequal to the first resonant frequency, when the beam conducts a second current from the current source.

22. The method of claim 21 comprising including the current source in a package that also includes the beam.

23. A system comprising:
a cavity formed within an insulating material;
a beam located in the cavity and having a long axis in a horizontal plane;
an interconnect to couple the beam to a current source;
a magnet coupled to the cavity; and
a polymer on the beam;
wherein (a) a vertical axis intersects the magnet, the cavity, and the beam; (b) when in a first state the beam and the polymer, which is not coupled to a chemical analyte, collectively have a first mass and resonate at a first resonant frequency when the beam conducts a first current from the current source; and (c) when in a second state the beam and the polymer, which is coupled to the chemical analyte, collectively have a second mass that is greater than the first mass and resonate at a second resonant frequency, unequal to the first resonant frequency, when the beam conducts a second current from the current source.

24. The system of claim 24, wherein the polymer is programmed to have an affinity to the chemical analyte.

25. The system of claim 24 including a logic module to produce a signal proportional to an amount of the chemical analyte coupled to the polymer in the second state.
